(19) United States
(12) Patent Application Publication
(10) Pub. No.: US 2025/0255618 A1
(43) Pub. Date: Aug. 14, 2025

(54) Surgical Handpiece System For Driving Surgical Pins And Related Accessories

(71) Applicant: Stryker Corporation, Portage, MI (US)

(72) Inventor: Patrick Roach, Portage, MI (US)

(73) Assignee: Stryker Corporation, Portage, MI (US)

(21) Appl. No.: 19/054,435

(22) Filed: Feb. 14, 2025

Related U.S. Application Data
(60) Provisional application No. 63/553,390, filed on Feb. 14, 2024.

(51) Int. Cl. A61B 17/16; A61B 17/00

(52) U.S. Cl. CPC A61B 17/1624; A61B 17/1626; A61B 17/1628; A61B 2017/00017

(57) ABSTRACT

A pin driver attachment system is provided. The system includes an input shaft to be coupled to a handpiece housing including a motor. The input shaft receives torque from the motor. The input shaft has a first drive portion having a first bore for connecting to a first surgical pin having a first diameter and a second drive portion having a second bore for connecting to a second surgical pin having a second diameter. The first bore is coaxial with and smaller than the second bore. First and second flexible couplers are secured to the input shaft and associated with the first and second bores. Each coupler has an annular shape and is movable between an engaged position and an unengaged position to secure and release the respective surgical pin to the respective bore. The first coupler has a smaller cross-sectional area than the second coupler.

### CROSS-REFERENCE TO RELATED APPLICATIONS

The subject application claims priority to and all the benefits of U.S. Provisional Patent Application No. 63/553,390, filed Feb. 14, 2024, the disclosure of which is incorporated by reference in its entirety herein.

### BACKGROUND

Conventional medical and surgical procedures routinely involve the use of surgical tools and instruments which allow surgeons to approach and manipulate surgical sites. By way of non-limiting example, rotary instruments for driving surgical pins such as a surgical handpiece assembly are commonly utilized in connection with orthopedic procedures to drive surgical pins into bone.

While surgical handpiece assemblies for driving surgical pins are routinely utilized to assist in the performance of a variety of different types of medical and/or surgical procedures, there is a need in the art to continuously improve such surgical handpiece assemblies.

### SUMMARY

A first aspect of the present disclosure provides a surgical pin driver attachment system. The pin driver attachment includes an input shaft to be removably coupled to a handpiece housing for receiving torque from a motor of the handpiece housing. The input shaft has a first drive portion having a first bore for connecting to a first surgical pin having a first diameter. The input shaft has a second drive portion having a second bore for connecting to a second surgical pin having a second diameter. The first bore is coaxial with the second bore and the first bore is smaller than the second bore. The pin driver attachment also includes first and second flexible couplers coupled to the input shaft and associated with the first and second bores, respectively. Each flexible coupler includes an annular shape and is movable between an engaged position to secure a surgical pin to the respective bore and an unengaged position to permit the respective surgical pin to be removed from the respective bore. The first flexible coupler defines a smaller cross-sectional area than the second flexible coupler.

A second aspect of the present disclosure provides a surgical power tool system for driving a surgical pin. The surgical power tool system includes a surgical handpiece assembly. The surgical handpiece assembly includes a handpiece housing, an electric motor positioned in the handpiece housing, a controller positioned in the handpiece housing, a battery removably coupled to the handpiece housing and configured to supply electrical power to the controller, an output drive coupled to the electric motor and configured to be rotated about an axis by the electric motor. The surgical power tool system also includes a pin driver attachment removably coupled to the surgical handpiece assembly. The pin driver attachment includes an input shaft to be removably coupled to the handpiece housing for receiving torque from the motor of the handpiece housing. The input shaft has a first drive portion having a first bore for connecting to a first surgical pin having a first diameter. The input shaft has a second drive portion having a second bore for connecting to a second surgical pin having a second diameter. The first bore is coaxial with the second bore and the first bore is smaller than the second bore. The pin driver attachment also includes first and second flexible couplers coupled to the input shaft and associated with the first and second bores, respectively. Each flexible coupler includes an annular shape and is movable between an engaged position to secure a surgical pin to the respective bore and an unengaged position to permit the respective surgical pin to be removed from the respective bore. The first flexible coupler defines a smaller cross-sectional area than the second flexible coupler.

### BRIEF DESCRIPTION OF THE DRAWINGS

Advantages of the present invention will be readily appreciated as the same becomes better understood by reference to the following detailed description when considered in connection with the accompanying drawings wherein:

FIG. 1 is a perspective view of a surgical system including a surgical handpiece and a surgical attachment.

FIG. 2 is another perspective view of the surgical system including the surgical handpiece and a proximal portion of the surgical attachment.

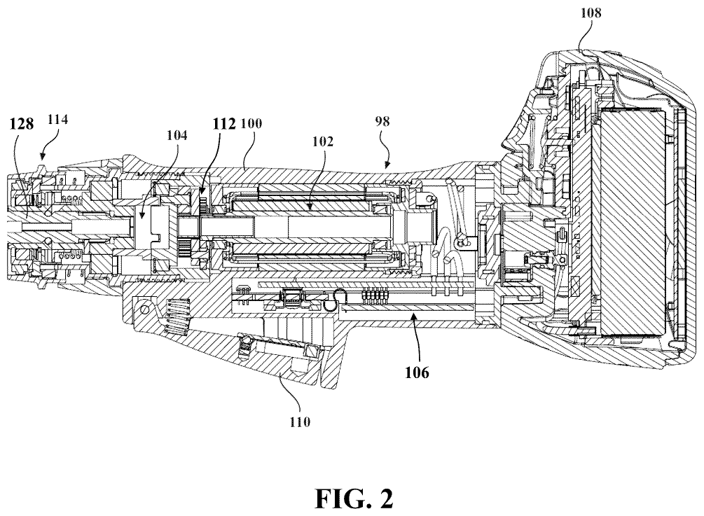

FIG. 3 is a perspective view of the surgical attachment.

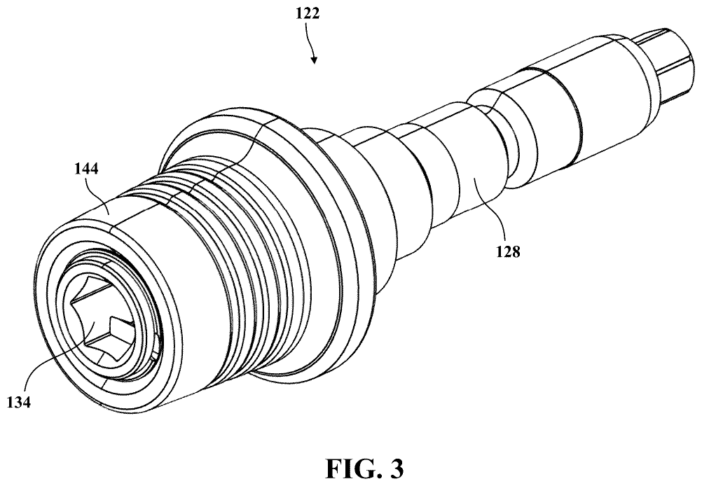

FIG. 4 is a section view of the surgical attachment.

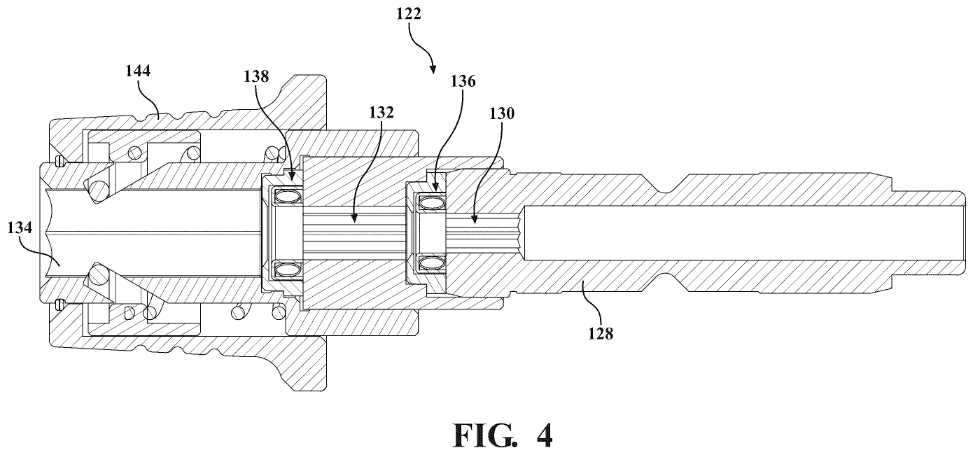

FIG. 5 is a detailed view of the surgical attachment of FIG. 4.

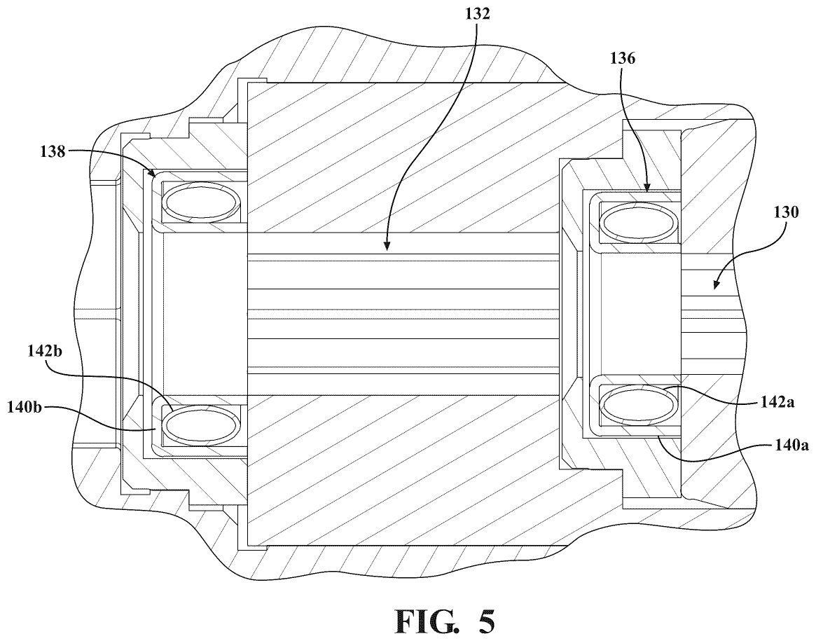

### DETAILED DESCRIPTION

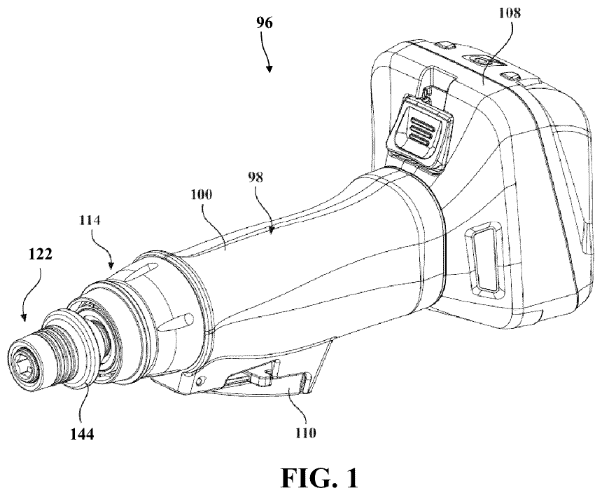

With reference to FIG. 1, a surgical power tool assembly 98 of a surgical system 96 is contemplated. The surgical power tool assembly 98 may be referred to as a surgical handpiece assembly or a surgical handpiece system. An exemplary surgical power tool assembly is described in U.S. Patent Publication No. 2016/0206327. The power tool assembly may include a surgical handpiece having a housing 100, an electric motor 102 positioned in the housing 100, and an output drive 104 operatively coupled to and actuated by the electric motor 102 such that the output drive 104 is rotated by the motor 102 about an output drive axis AX. The output drive 104 may be configured to engage any suitable surgical end effector such as a surgical pin or wire, or a drill bit, or a driver. The power tool assembly may further include a controller 106. The controller 106 may be optionally positioned within the housing 100. The surgical power tool assembly may optionally include a removable battery 108 that is configured to supply electrical power to the controller and to the electric motor 102. One battery that can be employed with this version of the invention is described in U.S. Patent App. Pub. No. 2007/0090788 published on Apr. 26, 2007, and herein incorporated by reference. The battery 108 may be configured to be coupled to the housing via a twist lock design. One suitable example of such a battery is described in U.S. Pat. No. 11,534,181, issued on Dec. 27, 2022, and herein incorporated by reference in its entirety. A trigger 110 may be movably coupled to the housing 100, and suitable trigger sensors may be included in the tool such that the controller 106 is configured to turn on the electric motor 102 and/or adjust the speed of the motor 102 and in turn the output drive 104 based on the extent that the trigger 110 is actuated and/or depressed. The output drive 104 may include a gearset 112 disposed between the motor 102 and a tool coupler 114 of the output drive 104 to increase torque available at the tool coupler 114. In some configurations, the tool coupler 114 may be used to secure surgical pins and/or drill bits to the handpiece, in other configurations, the tool coupler 114 may be used to secure one or more of the surgical attachments to the handpiece. In one configuration, the controller 106 is configured to regulate power drawn from a power source (e.g., the battery) based on user input on the trigger 110. In some configurations, the trigger 110 is pivotably coupled to the housing 100.

A range of surgical attachments may be used with the surgical power tool assembly 98 contemplated herein. One such surgical attachment is a pin driver attachment 122. The pin driver attachment 122 includes an input shaft 128 to be removably coupled to the output 104 of the power tool assembly 98 that defines features for receiving torque from the output drive 104 of the handpiece 98. The pin driver attachment may include a first drive portion having a first bore 130, a second drive portion having a second bore 132. In some configurations, the pin driver attachment also includes a third drive portion having a third bore 134. The first bore 130 may be coaxial with the second bore 132 and optionally the third bore 134. The first bore 130 may be smaller than (e.g., have a different or smaller cross-sectional area) the second bore 132 and the second bore 132 may be smaller than the third bore 134. The first bore 130 may include a plurality of flats for driving the drill bit or the surgical pin. The second bore 132 may also include a plurality of flats for driving a different sized drill bit or the surgical pin. In some configurations, the first and or second bores may include more flats or vertices where the flats meet than are disposed on the corresponding surgical pin or bit such that orienting the surgical pin or bit requires less rotation to align driving surfaces.

One or more flexible tool couplers 136, 138 are secured to the input shaft 128. The flexible tool couplers 136, 138 are movable between an engaged position and an unengaged position. In the engaged position, a drill bit or pin can be secured by the flexible tool couplers 136, 138. The third drive portion 134 may be used to drive a twist drill. A third tool coupler 144 may be slidably coupled to the surgical attachment to secure the twist drill to the third drive portion 134. The first and second drive portions 130, 132 may be used to drive two different sized surgical pins or bits. In the unengaged position, the drill bit or surgical pin can be released from the flexible tool couplers 136, 138. The flexible tool couplers 136, 138 are biased to the engaged position and are moveable to the unengaged position by the user applying axial force on the surgical pin or bit in opposition to a biasing force of the flexible tool coupler 136, 138 to permit a user to insert the surgical pin into the respective drive portions by insertion without requiring the user to manipulate an actuator or otherwise further manipulate a coupler to a released position. The biasing force of the flexible tool coupler 136, 138 is strong enough to keep the surgical pin or bit from falling out of the respective bore 130, 132 unintentionally. The user may remove the surgical pin from the pin driver attachment 122 by applying enough axial force in a distal direction to overcome the biasing force of the flexible tool coupler 136, 138. The first flexible tool coupler 136 defines a smaller cross-sectional area than the second flexible coupler 138. The different sized flexible tool couplers 136, 138 permit the user to use different sized pins in the same surgical procedure without changing surgical attachments.

In some configurations, the first and second bores 130, 132 may define fluted channels helically disposed around the associated bore 130, 132 to accommodate fluted surgical pins. In such a configuration, when a fluted surgical pin is engaged by the respective tool coupler 136, 138, the user must overcome friction associated with the helical surgical pin needing to rotate in the fluted channels while additionally overcoming the biasing force of the tool couplers 136, 138 to remove the fluted surgical pin axially from the pin driver attachment 122. In this configuration, the fluted surgical pin can assist with unintentional removal of the surgical pin from the associated drive portion when the user is attempting to remove the surgical pin from bone.

The first and second tool flexible couplers 136, 138 each comprise a cover 140a, 140b and a spring 142a, 142b disposed at least partially inside the cover 140a, 140b. The cover 140a, 140b may comprise a polymeric material. The spring 142a, 142b may comprise metal. The spring 142a, 142b is configured to bias inwardly to the engaged position to force a portion of the cover 140a, 140b into abutting contact with the surgical pin or bit to secure the surgical pin or bit to the respective driving portion. The first flexible tool coupler 136 may be disposed proximal the second drive portion 132 and distal the first drive portion 130. In some configurations, the first flexible coupler 136 is disposed proximal the first drive portion 130. In some configurations, the first flexible coupler 136 may be disposed within the first drive portion 130 such that the first drive portion includes flats for driving the surgical pin or bit that are disposed proximally and distally to the first flexible coupler 136. In some configurations, the second flexible coupler 138 may be disposed within the second drive portion 132 such that the second drive portion 132 includes flats for driving the surgical pin or bit that are disposed proximally and distally to the second flexible coupler 138. The flexible tool couplers 136, 138 may alternatively be referred to as active seals.

A method of driving pins in a robotically-assisted knee arthroplasty procedure is also contemplated. The method includes providing the battery to the surgical power tool assembly 98. The surgical attachment 122, such as the pin driver attachment 122, is coupled to the surgical power tool assembly 98. A hole is drilled in a femur, a tibia, or a patella of a patient with a first drill bit using the surgical attachment 122. A first pin is driven in the femur, the tibia, or the patella with the surgical attachment. A second pin is driven in one of the other of the femur, the tibia, and the patella with the surgical attachment. The first pin may have a different diameter than the second pin. The pins may be used to secure navigation trackers (not shown) to bones of the patient. Using the same surgical attachment 122 for each of the pins helps to make workflow more efficient by saving time during a procedure by not having to use multiple surgical attachments and reducing the number of components that require sterilization. Furthermore, the stack-up of components is smaller, which decreases the distance between where the pin/tool/drill bit is coupled to the surgical power tool assembly 98 and where the user grasps the surgical power tool assembly 98.

The motor 102 may be a brushless DC motor. The surgical tool 98 may include a 3-phase H-bridge to commutate the brushless DC motor. The H-Bridge may control the speed and direction of the powered motor.

Several configurations have been discussed in the foregoing description. However, the configurations discussed herein are not intended to be exhaustive or limit the invention to any particular form. The terminology which has been used is intended to be in the nature of words of description rather than of limitation.

What is claimed is:

1. A surgical pin driver attachment, the surgical pin driver attachment comprising:
an input shaft to be removably coupled to a handpiece housing for receiving torque from a motor of the handpiece housing, the input shaft having a first drive portion having a first bore for connecting to a first surgical pin having a first diameter and a second drive portion having a second bore for connecting to a second surgical pin having a second diameter, the first bore being coaxial with the second bore and the first bore being smaller than the second bore; and
first and second flexible couplers coupled to the input shaft and associated with the first and second bores, respectively, each flexible coupler comprising an annular shape and movable between an engaged position to secure a surgical pin to the respective bore and an unengaged position to permit the respective surgical pin to be removed from the respective bore;
wherein the first flexible coupler defines a smaller cross-sectional area than the second flexible coupler.

2. The surgical pin drive attachment of claim 1, wherein the first bore includes a plurality of flats, and the second bore includes a plurality of flats, and further comprising a surgical pin including at least four flats.

3. The surgical pin driver attachment of claim 1, wherein the first bore is proximal to the second bore.

4. The surgical pin driver attachment of claim 1, wherein the flexible couplers are biased to the engaged position.

5. The surgical pin driver attachment of claim 1, wherein the first and second flexible couplers each comprise a cover and a spring disposed at least partially inside the cover.

6. The surgical pin driver attachment of claim 5, wherein the cover comprises a polymeric material.

7. The surgical pin driver attachment of claim 1, wherein the input shaft further comprises a third drive portion for connecting to a twist drill.

8. The surgical pin driver attachment of claim 7, wherein the first drive portion defines a first cross-sectional area, the second drive portion defines a second cross-sectional area different from the first cross-sectional area, and the third drive portion defines a third cross-sectional area different from the first and second cross-sectional areas.

9. The surgical pin driver attachment of claim 1, wherein at least one of the first and second bores define helical flutes for receiving a fluted surgical pin.

10. A surgical power tool system for driving a surgical pin, the surgical power tool assembly comprising:
a surgical handpiece assembly comprising:
a handpiece housing,
an electric motor positioned in the handpiece housing,
a controller positioned in the handpiece housing,
a battery removably coupled to the handpiece housing and configured to supply electrical power to the controller,
an output drive coupled to the electric motor and configured to be rotated about an axis by the electric motor; and
a pin driver attachment removably coupled to the surgical handpiece assembly, the pin driver attachment comprising:
an input shaft removably coupled to the handpiece housing for receiving torque from the electric motor of the surgical handpiece, the input shaft having a first drive portion having a first bore for connecting to a first surgical pin having a first diameter and a second drive portion having a second bore for connecting to a second surgical pin having a second diameter, the first bore being coaxial with the second bore and the first bore being smaller than the second bore; and
first and second flexible couplers coupled to the input shaft and associated with the first and second bores, respectively, each flexible coupler comprising an annular shape and movable between an engaged position to secure a surgical pin to the respective bore and an unengaged position to permit the respective surgical pin to be removed from the respective bore;
wherein the first flexible coupler defines a smaller cross-sectional area than the second flexible coupler.

11. The surgical power tool system of claim 10, wherein the first bore includes a plurality of flats, and the second bore includes a plurality of flats, and further comprising a surgical pin including at least four flats.

12. The surgical power tool system of claim 10, wherein the first bore is proximal to the second bore.

13. The surgical power tool system of claim 10, wherein the flexible couplers are biased to the engaged position.

14. The surgical power tool system of claim 10, wherein the first and second flexible couplers each comprise a cover and a spring disposed at least partially inside the cover.

15. The surgical power tool system of claim 14, wherein the cover comprises a polymeric material.

16. The surgical power tool system of claim 10, wherein the input shaft further comprises a third drive portion for connecting to a twist drill.

17. The surgical power tool system of claim 16, wherein the first drive portion defines a first cross-sectional area, the second drive portion defines a second cross-sectional area different from the first cross-sectional area, and the third drive portion defines a third cross-sectional area different from the first and second cross-sectional areas.

18. The surgical pin driver attachment of claim 10, wherein at least one of the first and second bores define helical flutes for receiving a fluted surgical pin.
